(19) United States
(12) Patent Application Publication
(10) Pub. No.: US 2012/0016170 A1
(43) Pub. Date: Jan. 19, 2012

(54) PROCESS FOR PREPARING A CORE-LAYER MATERIAL HAVING GOOD MECHANICAL STRENGTH

(75) Inventors: Samy Ould-Chikh, Tassin La Demi Lune (FR); Antoine Fecant, Brignais (FR); Loic Rouleau, Charly (FR); Lars Fischer, Vienne (FR); Mehrdji Hemati, Pins-Justaret (FR)

(73) Assignee: IFP Energies nouvelles, RUEIL-MALMAISON CEDEX (FR)

(21) Appl. No.: 13/059,666

(22) PCT Filed: Aug. 19, 2009
(86) PCT No.: PCT/FR09/01007

§ 371 Date: Sep. 29, 2011

(30) Foreign Application Priority Data
Aug. 19, 2008 (FR) 08/04.639

(51) Int. Cl. C07C 5/05; B01J 23/42; B01J 21/04; B01J 37/08; B01J 37/16; B01J 37/025; B01J 37/02

(52) U.S. Cl. 585/273; 502/10

(57) ABSTRACT

A process is described for preparing a spherical material comprising a porous core coated with a continuous and homogeneous porous layer, the degree of attrition of said material being less than 20%. Said preparation process comprises a) bringing a bed of spherical particles constituting the core of said material into contact with a suspension containing an inorganic binder in order to form a solid having a pre-layer around said core; b) bringing the solid derived from step a) into contact, in a stream of hot air, with a powder constituted by spherical particles of an inorganic oxide and a suspension containing an inorganic binder and an organic binder in order to form a solid the core of which is coated with at least one continuous and homogeneous porous layer, the ratio of the (mass of anhydrous inorganic binder/volume of powder particles) being in the range 0.05 to 1 g.mL⁻¹; c) drying the solid derived from said step b); and d) calcining the solid derived from said step c).

### FIELD OF THE INVENTION

The present invention relates to the field of materials comprising a core coated with at least one continuous and homogeneous outer layer. More precisely, it relates to a novel process for the preparation of a core-layer material with a view to using said material as catalyst supports in selective hydrogenation reactions.

### PRIOR ART

Core-layer materials are widely described for catalysis and/or adsorption. Such materials are, for example, constituted by a layer containing a catalytically active phase and a catalytically inactive core, and can in particular produce a significant gain in selectivity in reactions necessitating high material and/or heat transfer. Materials with an inert core and catalytically active layer are, for example, described in reactions for the dehydrogenation of paraffinic cuts (U.S. Pat. No. 6,177,381, U.S. Pat. No. 6,486,370), the hydroisomerisation of paraffinic cuts (EP 0,542,528, U.S. Pat. No. 5,200,382), the aliphatic alkylation of gasoline cuts (WO 98/14274), the hydrodesulphurization of naphtha containing olefins (WO 2006/009773), the hydrodesulphurization, hydrodenitrogenation or hydrodemetallization of hydrocarbons (U.S. Pat. No. 4,255,253), the alkylation of aromatic cuts (U.S. Pat. No. 4,283,583, U.S. Pat. No. 6,376,730), the isomerisation of aromatic cuts (U.S. Pat. No. 7,297,830), the selective hydrogenation of unsaturated hydrocarbons (U.S. Pat. No. 6,992,040, U.S. 2003/0036476), the oxidation of acrolein to acetic acid (U.S. Pat. No. 5,677,261), the production of alkenyl carboxylate (vinyl acetate) from carboxylic acid (acetic acid), olefin (ethylene) and oxygen (air) (WO 2007/008288), the oxychlorination of olefins or aromatics (U.S. Pat. No. 4,460,699), oxide of nitrogen reduction (U.S. Pat. No. 4,585,632), the oxidation of hydrogen to water (WO 2004/035201), and the production of hydrocarbons from synthesis gas (U.S. Pat. No. 7,087,191, U.S. 2005/0245621).

These materials are prepared by bringing elements of the layer into contact with a support acting as a core. The core may be inert and correspond, for example, to a refractory oxide support such as cordierite or alpha alumina. The core may be catalytically active and correspond, for example, to a catalyst constituted by an active phase dispersed in a porous alumina matrix. The core may be adsorbant.

The elements of the layer are intended to form the layer and may be taken up entirely into aqueous suspension (U.S. Pat. No. 6,177,381) then brought into contact with the core. They may be partially in suspension and partially in the form of a powder (U.S. Pat. No. 5,677,261) then brought into contact with the core. Additives are sometimes added to encourage certain characteristics, for example wetting the external periphery of the core by the suspension or the mechanical characteristics of the layer. Organic polyvinyl alcohol type additives, glycerol or cellulose derivatives are, for example, added to the suspension to enhance the wettability thereof on the outer surface of the core and the attrition resistance of the core-layer materials obtained (U.S. Pat. No. 6,486,370). Fibres, generally inorganic, may also be added to the elements to improve the impact strength (WO 2007/008288). The catalytically active layers are comparable to conventional catalysts and are, for example, formed by a metal phase and/or zeolitic phase dispersed in a porous alumina matrix. They are, for example, obtained from a suspension of elements of the layer comprising an alumina sol acting as the binder, dissolved metallic salts intended to form a catalytically active phase, alumina oxide which may optionally support a catalytically active metallic phase or zeolite. Other metallic phases may be added after the contacting step.

The elements of the layer are brought into contact with the elements constituting the core of the material by, for example, immersing the core in a liquid containing the elements of the layer (U.S. Pat. No. 4,460,699), by spraying a liquid containing the elements of the layer onto the core or by spraying a liquid and introducing a solid, comprising the elements of the layer, onto the core, in a simultaneous or consecutive step.

In order to be used widely in industrial processes, these materials must be obtained by a simple technique of depositing the layer and reduced in a number of steps. It is also desirable to obtain a material having a continuous and homogeneous layer, without cavity, crazing or cracking defects, which is highly cohesive and which adheres strongly to the surface of the core in order to resist high compressive and attrition stresses encountered in the majority of industrial applications.

### Aim And Advantage of the Invention

The present invention concerns a novel process for preparing a spherical material comprising at least one porous core coated with at least one continuous and homogeneous porous layer, the degree of attrition of said material, measured using the Spence method, being less than 20%, said preparation process comprising at least the following steps:

a) bringing at least one bed of spherical particles constituting the core of said material into contact with at least one suspension containing at least one inorganic binder in order to form a solid having a pre-layer surrounding said core;

b) bringing the solid derived from said step a) into contact in a stream of hot air with at least one powder constituted by spherical particles of at least one inorganic oxide and at least one suspension containing at least one inorganic binder and at least one organic binder in order to form a solid the core of which is coated with at least one continuous and homogeneous porous layer, the ratio of the (mass of anhydrous inorganic binder/volume of powder particles) being in the range 0.05 to 1 g.mL⁻¹;

c) drying the solid derived from said step b);

d) calcining the solid derived from said step c).

The material obtained for carrying out this novel preparation process has excellent attrition resistance, providing evidence of its very high mechanical strength. It resists compressive and shear stresses encountered in the majority of industrial applications. Surprisingly, said core-layer material prepared using the process of the invention is more resistant to compression or to attrition than those prepared using prior art preparation processes, especially those employing pre-wetting, an organic additive, or bringing into contact in a fluidized bed.

Further, said material obtained by carrying out said novel preparation process has a continuous and homogeneous layer, with few or even no defects of the cavity, crazing or crack type. Said layer is highly cohesive and adheres strongly to the surface of the core of said material. The prior formation of a pre-layer around the core results in very good keying of the particles of the powder constituting the layer to the core of the material. Further, another advantage of the process for the preparation of the core-layer material of the invention is that it employs a simple technique for depositing the layer and is thus highly economical.

The present invention also pertains to the preparation of a catalyst using the core-layer material prepared using the process of the invention as a catalyst support. At least one metal from group VIII and optionally at least one additional metal from group IB is (are) impregnated into the pores of the layer of the core-layer material. The catalyst obtained thereby is employed in a process for selective hydrogenation of polyunsaturated compounds present in hydrocarbon cuts. The catalyst comprising the core-layer material prepared using the process of the invention can produce better catalytic performances as regards the selective hydrogenation of polyunsaturated compounds, especially in terms of activity, than a catalyst comprising a core-layer material that is less resistant to attrition and a catalyst comprising a core-layer material in which the layer is discontinuous and less homogeneous.

### Description of the Invention

The present invention pertains to a process for preparing a spherical material comprising at least one porous core coated with at least one continuous and homogeneous porous layer, the degree of attrition of said material, measured using the Spence method, being less than 20%, said preparation process comprising at least the following steps:

a) bringing at least one bed of spherical particles constituting the core of said material into contact with at least one suspension containing at least one inorganic binder in order to form a solid having a pre-layer surrounding said core;

b) bringing the solid derived from said step a) into contact in a stream of hot air with at least one powder constituted by spherical particles of at least one inorganic oxide and at least one suspension containing at least one inorganic binder and at least one organic binder in order to form a solid the core of which is coated with at least one continuous and homogeneous porous layer, the ratio of the (mass of anhydrous inorganic binder/volume of powder particles) being in the range 0.05 to 1 g.mL⁻¹;

c) drying the solid derived from said step b);

d) calcining the solid derived from said step c).

The porous core present in the spherical material prepared using the process of the invention is constituted by particles with a spherical shape which have a narrow size distribution. The mean size or mean diameter of the distribution is in the range 0.1 to 10 mm, preferably in the range 0.3 to 7 mm and more preferably in the range 0.5 to 5 mm. The smallest diameter of the distribution is more than 0.5 times the mean diameter, preferably more than 0.8 times the mean diameter and still more preferably more than 0.9 times the mean diameter. The largest diameter of the distribution is less than 2 times the mean diameter, preferably less than 1.2 times the mean diameter and more preferably less than 1.1 times the mean diameter. The diameter of the particles constituting the core is preferably determined by optical microscopy or by scanning electron microscopy. The porous core is advantageously inert as regards adsorption or catalysis, i.e., it does not perform as an adsorbant or catalyst. It is, for example, constituted by alumina, zirconia, silica, titanium oxide, cordierite or a mixture of these compounds. The porous core may also be constituted by a zirconia, a molecular sieve or a mixture of said compounds and thus ensure an adsorbant function. The porous core may also be formed by a metallic phase and/or zeolitic phase dispersed in an alumina matrix and thus be catalytically active. Preferably, the core of the material prepared using the process of the invention is inert as regards adsorption or catalysis and is highly preferably constituted by alumina.

The specific surface area, total pore volume and pore size distribution of the porous core constituted by spherical particles may be highly variable as a function of the chemical nature of the particles constituting said core. The specific surface area is measured by nitrogen physisorption. The total pore volume and the pore size distribution are measured by mercury porosimetry. For an inert core, for example of alumina, the specific surface area is less than 50 m²/g, preferably less than 30 m²/g and more preferably less than 20 m²/g. The mean pore size is more than 50 nm, preferably more than 85 nm and more preferably more than 125 nm. The total pore volume is in the range 0.1 to 1 mL/g.

The porous layer which coats the core of said material prepared in accordance with the process of the invention is continuous, i.e. not fragmented or segmented, and homogeneous, i.e. it has a chemical composition which is close to and preferably identical from one location to another of the layer as well as with a thickness which is close to and preferably identical from one location to another of the layer. The differences in thickness from one location to another of the layer are less than 20% of the mean thickness value, preferably less than 15% of the mean thickness value and still more preferably less than 10% of the mean thickness value. The mean thickness value of the layer is in the range 0.005 to 1 mm, preferably in the range 0.007 to 0.7 mm and still more preferably in the range 0.01 to 0.5 mm. The thickness of the layer is measured by scanning electron microscopy on a polished section of the material. The deviations in the chemical elements present in the layer from one location to another of said layer may be detected in the case of a layer containing several elements, in particular for a layer constituted by a catalytically active phase in a porous inorganic matrix. These differences in chemical element contents are less than 20% of the mean value, preferably less than 15% of the mean value and still more preferably less than 10% of the mean value. The quantity of chemical elements in the layer is measured on a polished section of the material prepared using the process of the invention by X ray microanalysis (EDX) coupled to scanning electron microscopy (SEM) or Castaing microprobe (EPMA).

The porous layer is advantageously inert as regards adsorption or catalysis, i.e. it does not have any real performance in adsorption or in catalysis. It is, for example, constituted by alumina, zirconia, silica, titanium oxide or a mixture of these compounds, preferably alumina. Said porous layer may also be constituted by a zeolite, a molecular sieve or a mixture of said compounds and thereby provide an adsorbent function. Said porous layer may also be formed by a metallic and/or zeolitic phase dispersed in an alumina matrix and thus be catalytically active. Said porous layer may also be formed from a material with pores of a calibrated size corresponding to a membrane. As an example, it may be alumina, zirconia, silica, titanium oxide, zeolite, a molecular sieve or a mixture of said compounds. Preferably, said porous layer is inert as regards adsorption or catalysis and is preferably constituted by alumina. In accordance with the invention, when the core or the layer are constituted by alumina, the phase of the alumina constituting the particles of the core differs from that of the layer; for example, the core is constituted by an alpha alumina and the layer is constituted by a gamma alumina.

Said porous layer is highly cohesive, i.e. it is formed by particles with high mutual cohesion, and it adheres strongly to the core of said material prepared using the process of the invention. The cohesion and adhesion resistance of said core-layer material prepared using the process of the invention is measured by means of an attrition test using the Spence method (J. F. Le Page, Catalyse de contact: conception, preparation et mise en oeuvre des catalyseurs industriels [Contact catalysis: design, preparation and applications of industrial catalysts], Technip, 1978, page 225). The attrition test used to determine the degree of attrition of the material prepared using the process of the invention is carried out as follows: 25 g of material prepared using the process of the invention is caused to rotate in a stainless steel tube with a 36 mm diameter and length of 305 mm. The tube rotates about a perpendicular axis at 25 rpm for 1 h. The product is recovered and sieved with a normalized sieve, NFX-11-504/ISO-3310-2, the opening diameter of which is a function of the diameter of the material. In particular, in the context of the present invention, a sieve is selected with a mesh aperture diameter corresponding to two thirds of the mean diameter of the material prepared using the process of the invention and more preferably, a sieve is selected with an opening diameter of 1 mm. Sieving means that the mass of fines produced during rotation can be measured. The attrition resistance of the material prepared using the process of the invention is evaluated by the degree of attrition which is calculated by the ratio of the (mass of fines produced during rotation)/(mass of layer of material introduced into the test)×100. Surprisingly, the degree of attrition of the material prepared using the process of the present invention is small: it is less than 20%, preferably less than 10% and more preferably less than 5%. A low degree of attrition as presented by the material prepared using the process of the present invention is advantageous as it is demonstrative of the good mechanical strength of said material.

In accordance with step a) of the process for preparing the material of the invention, a suspension containing at least one inorganic binder and optionally at least one organic binder introduced into a solvent is (are) brought into contact with at least one bed of spherical particles constituting the core of said material. The inorganic binder may be a salt, an alkoxide, a hydroxide, an oxyhydroxide or an oxide which is soluble or dispersible in the solvent. It may, for example, be a sol of alumina, a sol of zirconia, a sol of silica, a sol of titanium oxide or a mixture of said sols. Said inorganic binder produces an increased rupture strength of the layer core interface of said material obtained at the end of said step d) of the preparation process of the invention. The organic binder, preferably present in the suspension brought into contact with at least one bed of spherical particles constituting the core of said material, may be a compound which is soluble or dispersible in the solvent and which decomposes after calcining in accordance with said step d). It may, for example, be polyvinyl alcohol, glycerol, or a cellulose derivative (methyl cellulose, carboxylmethyl cellulose, hydroxycellulose or hydroxypropylmethyl cellulose). It can limit crazing or cracking type defects which may form in the pre-layer during step c) and can thus increase the attrition resistance of the material after step d). The solvent may be water or a low molecular weight organic compound containing fewer than 6 carbon atoms per molecule and selected from an alcohol, a ketone, an ester, an ether or a mixture of said compounds, preferably water, ethanol or acetone or a mixture of said compounds, and more preferably water. Said step a) results in the formation of a pre-layer around each of the spherical particles constituting the core of material prepared using the process of the invention. Deposition of said pre-layer around each of the particles constituting the core of said material is preferably carried out in the absence of any air flow. Thus, at the end of said step a), the pre-layer is not dried and remains moist, encouraging better keying of the particles of the powder constituting the layer onto the core of material. The pre-layer is formed in order to cover the outer surface of each of the particles constituting the core of said material over a predetermined thickness and to fill the openings of the pores of said particles to a predetermined depth. The thickness of the pre-layer on the outer surface of the core is in the range 0.1 to 10 μm, preferably in the range 0.3 to 5 μm, and more preferably in the range 0.5 to 3 μm. The depth of the pre-layer in the pore openings of the porous core constituted by spherical particles is in the range 0.3 to 30 μm, preferably in the range 1 to 15 μm, and still more preferably in the range 1.5 to 10 μm. The presence of defects, the evaluation of the thickness and the depth of the pre-layer are determined by scanning electron microscopy carried out on a polished section of the solid derived from said step d), preferably without carrying out said step b). In accordance with said step a) of the process for the preparation of the material of the invention, the proportion of inorganic binder is defined so as to comply with the thickness and the depth of the pre-layer. The ratio of the (mass of anhydrous inorganic binder)/(mass of core)×100 is in the range 0.1% to 10%, preferably in the range 0.2% to 5% and more preferably in the range 0.3% to 3%. The proportion of organic binder, when present, is defined such that the ratio of the (mass of organic binder)/(mass of anhydrous inorganic binder)×100 is in the range 0.5% to 10%, preferably in the range 0.7% to 7%, and more preferably in the range 0.5% to 5%. The concentration by weight of inorganic binder in the solvent is defined such that the ratio of the (mass of anhydrous inorganic binder)/(mass of anhydrous inorganic binder+mass of solvent)×100 is in the range 0.2% to 20%, preferably in the range 0.4% to 10% and more preferably in the range 0.6% to 6%. The pre-layer is deposited using any technique known to the skilled person, advantageously by a simple technique, in particular by spraying the suspension onto a bed of spherical particles constituting the core of said material contained in an inclined rotating plate or in a rotating drum and executing a cascade movement (P. J. Sherrington, R. Oliver, Granulation, Heyden, 1981, p 62). Spraying of the suspension may be continuous or discontinuous. The conditions for deposit of the pre-layer, in particular the mean spray rate and the concentration of inorganic binder of the suspension, are selected so as to preserve cascade type movement throughout step a) and to ensure the formation of the pre-layer over a depth in the pore openings and over a thickness of the outer surface of the particles constituting the core as defined above, without cracking or crazing type defects.

In accordance with step b) of the process for the preparation of the material of the invention, at least one powder constituted by spherical particles of at least one inorganic oxide and at least one suspension containing at least one inorganic binder and at least one organic binder introduced into a solvent are deposited on the outer surface of the pre-layer formed around each of the particles of the core in step a) to form a continuous and homogeneous layer. The particles of the powder have a spherical shape and a calibrated size distribution which depends on the thickness of the layer. The shape of said particles is analyzed by scanning electron microscopy. The size distribution of said particles is measured by laser diffraction granulometry based on Mie's diffraction theory Mie (G. B. J. de Boer, C. de Weerd, D. Thoenes, H. W. J. Goossens, Part. Charact. 4 (1987) 14-19). The distribution of the granulometry of the powder particles is represented by the dimension Dv, X, defined as being the diameter of the equivalent sphere such that the size of X% by volume of the particles is less than said diameter. More precisely, the granulometric distribution of said particles is represented by the three dimensions Dv10, Dv50 and Dv90. The median diameter Dv50 is smaller than or equal to 0.2 times the thickness of the layer coating the core of said material prepared using the process of the invention, the thickness of said layer being measured after step d) of the process of the invention, preferably less than 0.15 times the thickness of the layer measured after said step d) and more preferably less than or equal to 0.1 times the thickness of the layer measured after said step d). The diameter Dv10 is more than or equal to 0.1 times the median diameter Dv50 and preferably more than or equal to 0.2 times the median diameter Dv50 and more preferably more than or equal to 0.5 times the median diameter Dv50. The diameter Dv90 is less than or equal to 10 times the median diameter and preferably less than or equal to 5 times the median diameter and more preferably less than or equal to 2 times the median diameter. The inorganic oxide constituting the spherical particles of said powder is particularly selected from alumina, zirconia, silica, titanium oxide and a mixture of at least one of said oxides.

The inorganic binder present in the suspension used to carry out said step b) of the process for the preparation of the material of the invention may be a salt, an alkoxide, a hydroxide, an oxyhydroxide or an oxide which is soluble or dispersible in the solvent employed in the suspension. It may, for example, be a sol of alumina, a sol of zirconia, a sol of silica, a sol of titanium oxide or a mixture of said sols. Said inorganic binder produces solidification of said layer coating the core of the material prepared using the process of the invention. The organic binder present in the suspension used to carry out said step b) of the preparation process of the invention may be a compound which is soluble or dispersible in the solvent used in the suspension. It decomposes after the calcining step of step d) of the preparation process of the invention. It limits crazing or cracking defects formed in the layer during drying step c) and thus increases the attrition resistance of the material prepared using the process of the invention. It may, for example, be a polyvinyl alcohol, glycerol, or a cellulose derivative (methyl cellulose, carboxylmethyl cellulose, hydroxycellulose or hydroxypropylmethyl cellulose). The solvent may be water or a low molecular washing organic compound containing fewer than 6 carbon atoms per molecule and selected from an alcohol, a ketone, an ester, an ether or a mixture of said compounds, and preferably water, ethanol or acetone or a mixture of these compounds and more preferably water. The layer is formed in order to cover the outer surface of the pre-layer in a continuous and homogeneous manner.

The proportion of particles constituting the powder introduced to carry out said step b) of the process for the preparation of the material of the invention is selected so as to obtain a thickness of the layer as defined above in the present description, namely a thickness in the range 0.005 to 1 mm, preferably in the range 0.007 to 0.7 mm and more preferably in the range 0.01 to 0.5 mm. The presence of defects and the assessment of the thickness of the layer are determined by scanning electron microscopy on a polished section of the material obtained at the end of said step d) of the preparation process of the invention. The ratio of the (mass of particles of powder)/(mass of particles of core)×100 is in the range 1% to 100%, preferably in the range 5% to 50% and more preferably in the range 3% to 30%. The proportion of inorganic binder is optimal when the particles of the powder present in the layer are close, preferably touching, and when the interparticulate voids created between the powder particles are filled with said inorganic binder with no residual porosity and with no crazing or cracking defects or segmentation of the layer after step d). This optimum point is determined by scanning electron microscopy on a polished section of the material obtained at the end of said step d). It may also be determined by the attrition test on the material obtained following said step d) and corresponds to the maximum attrition resistance. The ratio of the (mass of anhydrous inorganic binder)/(volume of particles of powder) is in the range 0.05 to 1 g/mL, preferably in the range 0.1 to 0.5 g/mL and more preferably in the range 0.15 to 0.35 g/mL. The proportion of organic binder in the suspension is defined such that the ratio of the (mass of organic binder)/(mass of anhydrous inorganic binder)×100 is in the range 0.5% to 10%, preferably in the range 0.5% to 7%, and more preferably in the range 0.7% to 5%. The concentration by weight of inorganic binder in the solvent employed in the suspension is defined such that the ratio of the (mass of anhydrous inorganic binder)/(mass of anhydrous inorganic binder +mass of solvent)×100 is in the range 0.2% to 20%, preferably in the range 0.4% to 10% and more preferably in the range 0.6% to 6%. The layer coating the core is deposited using any technique which is known to the skilled person, advantageously by a simple technique, especially by spraying, in a stream of hot air, of the suspension onto the particles of solid derived from said step a) contained in an inclined rotating plate or in a rotating drum and executing a cascade type movement. The stream of hot air may be continuous or it may be intermittent during the spray period. Spraying the suspension and introducing the particles of powder may be continuous or discontinuous. The conditions for deposition, in particular the mean flow rate of the powder particles, the mean spray rate, the concentration of inorganic binder of the suspension, the temperature of the hot air are defined so as to preserve cascade type movement throughout step b) and to ensure the formation of the continuous layer with a homogeneous thickness and composition over the outer surface of the pre-layer with no cracking and crazing defects. The temperature of the hot air stream is in the range 50° C. to 200° C., preferably in the range 60° C. to 150° C.

In step c) of the process for the preparation of the material of the invention, drying is carried out using techniques known to the skilled person, to evaporate the solvent in a controlled manner and to limit the crazing, cracking or segmentation type defects of the layer and to carry out initial solidification of the layer. Drying is in particular carried out at a temperature in the range 25° C. to 200° C., preferably in the range 30° C. to 150° C. for a period in the range 1 h to 80 h, preferably in the range 2 h to 12 h. It is carried out under vacuum, in ambient air or in moist air, with a water vapour content of 10% to 100% by volume. Highly preferably, drying is carried out at a temperature in the range 30° C. to 150° C. for a period of 2 h to 12 h in ambient air.

In accordance with step d) of the process for the preparation of the material of the invention, calcining is carried out using techniques which are known to the skilled person for decomposing the organic products introduced, revealing the pores of the material, calibrating the size of the pores of the layer formed and carrying out final solidification of the layer. Calcining is in particular carried out at a temperature in the range 400° C. to 1000° C., preferably in the range 450° C. to 800° C., for a period of 1 h to 12 h, in ambient air or moist air with a concentration of water vapour of 0 to 80%. More preferably, calcining is carried out at a temperature in the range 450° C. to 800° C. in ambient air.

The concatenation of steps a), b), c) and d) described above may be carried out a single time or multiple times with the same layer precursors to obtain a continuous and homogeneous single layer. This concatenation may be carried out multiple times with different precursors in order to obtain several continuous and homogeneous layers. This concatenation is preferably carried once or multiple times with the same precursors and more preferably a single time.

The present invention also pertains to a process for the preparation of a catalyst from a material prepared using the process described above in the present description. Following the preparation of the core-layer material in accordance with the process of the invention described above, a catalytically active phase is introduced into the porosity of the layer coating the core of said material which then acts as a catalyst support, using impregnation techniques which are known to the skilled person. The catalytically active phase comprises at least one metal from group VIII of the periodic classification of the elements and advantageously at least one additional metal from group IB. Said metal from group VIII is preferably selected from nickel, palladium and platinum and a mixture of at least two of said metals; preferably, said metal from group VIII is palladium. Said metal from group IB is selected from copper, silver and gold. The quantity by weight of metal from group VIII of the catalyst prepared from the material prepared in accordance with the process of the invention described above in the present description is in the range 0.01% to 20% by weight. In particular, when said metal from group VIII is palladium, its content by weight is in the range 0.05% to 2% by weight. When it is present, the weight content of the metal from group IB of the catalyst prepared from a material prepared in accordance with the process of the invention described above in the present description is in the range 0.01% to 2% by weight. The catalyst obtained is in the form of beads.

More precisely, the process for the preparation of the catalyst of the invention comprises the following steps:

e) impregnating the material derived from said step d) with at least one solution of at least one precursor of at least one metal from group VIII;

f) drying the solid derived from said step e);

g) calcining the solid derived from said step f); and

h) reducing the catalyst derived from said step g).

In accordance with said step e) of the process for the preparation of the catalyst of the invention, the core-layer material derives from said step d) of the process for the preparation of the invention described above in the present description may be impregnated by dry impregnation, by impregnation in excess or in shortfall, in static or dynamic mode. Preferably, dry impregnation is carried out in a bowl granulator. Impregnation may be carried out in one or more successive impregnation steps. Said step e) is carried out at a temperature in the range 5° C. to 40° C., preferably in the range 15° C. to 35° C.

The concentration of the solution of at least one precursor of at least one metal from group VIII, preferably a palladium precursor, is adjusted as a function of the quantity by weight of metal from group VIII desired in the catalyst. Preferably, said solution is an aqueous solution and the precursor salt of said metal from group VIII is generally selected from the group constituted by chlorides, nitrates and sulphates of metallic ions. When, as is preferred, the metal is palladium, the palladium precursor salt is advantageously palladium nitrate. In accordance with said step f) of the catalyst preparation process of the invention, the solid derived from said step e) is dried in order to eliminate all or a portion of the water introduced during impregnation of at least one metal from group VIII. Drying is carried out in air or in an inert atmosphere (for example nitrogen), preferably at a temperature in the range 50° C. to 250° C., more preferably in the range 70° C. to 200° C.

In accordance with said step g) of the process for the preparation of the catalyst of the invention, the dried solid derived from said step f) is calcined in air. The calcining temperature is generally in the range 250° C. to 900° C., preferably in the range from approximately 300° C. to 500° C. the calcining period is generally in the range 0.5 hours to 5 hours.

The calcined catalyst obtained at the end of said step g) undergoes a step h) for reduction at a temperature in the range 50° C. to 500° C., preferably in the range 80° C. to 180° C. The reduction is carried out in the presence of a reducing gas comprising in the range 25% by volume to 100% by volume of hydrogen, preferably 100% by volume of hydrogen. The hydrogen is optionally supplemented with an inert gas for the reduction, preferably argon, nitrogen or methane. The reduction period is generally in the range 1 to 15 hours, preferably in the range 2 to 8 hours. The hourly space velocity (HSV) is generally in the range 150 to 1000, preferably in the range 300 to 900 litres of reducing gas per hour per litre of catalyst.

When, in addition to at least one metal from group VIII, the catalyst comprises at least one additional metal from group IB, after said step f), a step e′) is carried out for impregnation of said solid derived from said step f) with at least one solution of at least one precursor of at least one metal from group IB. Said step e′) is carried out under the same operating conditions as those of said step e). Preferably, said solution of said precursor is an aqueous solution and the precursor salt of said metal from group IB is generally selected from the group constituted by chlorides, nitrates and sulphates of metallic ions. When, as is preferable, the metal is gold or silver, the precursor salts are chloroauric acid or silver nitrate respectively.

This impregnation step is followed by drying the solid impregnated with at least one metal from group IB under the conditions given for step f). Next, said solid undergoes said calcining step g).

The present invention also pertains to a process for selective hydrogenation, comprising bringing a hydrocarbon feed containing at least one polyunsaturated compound into contact with at least said catalyst prepared using the process described above in the present description.

Said hydrocarbon feed containing at least one polyunsaturated compound is advantageously selected from the group constituted by cuts derived from catalytic cracking, the C3 cut derived from steam cracking, the C4 cut derived from steam cracking, the C5 cut derived from steam cracking and gasolines derived from steam cracking, also termed pyrolysis gasolines. Said cuts contain compounds comprising acetylene functions, diene functions and/or olefin functions.

The use of the catalyst prepared in the process of the invention described above in the present description and the conditions for its use in the selective hydrogenation process will be adapted by the user to the technology employed in a manner which is known to the skilled person.

Processes for the conversion of hydrocarbons such as steam cracking or catalytic cracking are operated at high temperature and produce a wide variety of unsaturated molecules such as ethylene, propylene, straight chain butenes, isobutene, pentenes as well as unsaturated molecules containing up to approximately 15 carbon atoms. Polyunsaturated compounds are also formed and in particular acetylene, propadiene and methylacetylene (or propyne), 1,2- and 1,3-butadiene, vinyl acetylene and ethyl acetylene as well as other polyunsaturated compounds with a boiling point corresponding to the gasoline fraction C5+. The ensemble of said polyunsaturated compounds must be eliminated in order to allow the use of different cuts in which they are contained in petrochemical processes such as in polymerization units.

Thus, for example, the C3 cut from steam cracking may have the following mean composition: of the order of 90% by weight of propylene, of the order of 3% to 8% by weight of propadiene and methyl acetylene, the remainder essentially being propane. In certain C3 cuts, between 0.1% and 2% by weight of C2 and C4 compounds may also be present. The specifications concerning the concentrations of said polyunsaturated compounds for petrochemical and polymerization units are very low: 20-30 ppm by weight of MAPD (methyl acetylene and propadiene) for propylene when a C3 cut is to be used in a petrochemicals unit and less than 10 ppm by weight or even down to 1 ppm by weight when a C3 cut is to be used in a polymerization unit.

A C4 cut from steam cracking presents, for example, the following mean molar composition: 1% of butane, 46.5% of butane, 51% of butadiene, 1.3% of vinyl acetylene (VAC) and 0.2% of butyne. In certain C4 cuts, between 0.1% and 2% by weight of C3 and C5 compounds may also be present. The specifications concerning the concentrations of polyunsaturated compounds are severe: a diolefins content of strictly less than 10 ppm by weight for a C4 cut used in a petrochemicals or polymerization unit.

A C5 cut derived from steam cracking has, for example, the following mean composition by weight: 21% of pentanes, 45% of pentenes, 34% of pentadienes.

The selective hydrogenation process of the invention can eliminate polyunsaturated compounds from the C3 to C5 oil cuts cited above by the conversion of the most unsaturated compounds into the corresponding alkenes, avoiding total saturation and thus the formation of the corresponding alkanes.

Selective hydrogenation of C3, C4 and C5 cuts may be carried out in the gas phase or in the liquid phase, preferably in the liquid phase. A liquid phase reaction can reduce energy costs and increase the service life of the catalyst.

For a liquid phase reaction, the pressure is generally in the range 1 to 3 MPa, the temperature is in the range 2° C. to 50° C. and the hydrogen/(polyunsaturated compounds to be hydrogenated) molar ratio is in the range 0.1 to 4, preferably in the range 1 to 2. The HSV (feed flow rate per volume of catalyst) is in the range 10 to 50 h⁻¹.

For a gas phase hydrogenation reaction, the pressure is generally in the range 1 to 3 MPa, the temperature is in the range 40° C. to 120° C., the hydrogen/(polyunsaturated compounds to be hydrogenated) molar ratio in the range 0.1 to 4, preferably in the range 1 to 2, and the HSV (flow rate of feed per volume of catalyst) is in the range 500 h⁻¹ to 5000 h ⁻¹.

In accordance with a preferred implementation of the selective hydrogenation process of the invention, the hydrocarbon feed containing at least one polyunsaturated compound brought into contact with the catalyst prepared in accordance with the process of the invention is a gasoline derived from steam cracking. Said gasoline is termed pyrolysis gasoline. Pyrolysis gasoline corresponds to a cut with a boiling point generally in the range 0° C. to 250° C., preferably in the range 10° C. to 220° C. This feed generally comprises the C5-C12 cut with traces of C3, C4, C13, C14 and C15 compounds (for example in the range 0.1% to 3% by weight for each of said cuts).

As an example, a feed formed from a pyrolysis gasoline generally has the following composition by weight: 8% to 12% by weight of paraffins, 58% to 62% by weight of aromatic compounds, 8% to 10% by weight of mono-olefins, 18% to 22% by weight of diolefins and 20 to 300 ppm of sulphur.

In the case of selective hydrogenation of pyrolysis gasoline, the hydrogen/(polyunsaturated compounds to be hydrogenated) molar ratio is generally in the range 1 to 2, the temperature is generally in the range 40° C. to 200° C., preferably in the range 50° C. to 180° C., the hourly space velocity (corresponding to the volume of hydrocarbon per volume of catalyst per hour) is generally in the range 0.5 h⁻¹ to 10 h⁻¹, preferably in the range 1 h⁻¹ to 5 h⁻¹ and the pressure is generally in the range 1.0 MPa to 6.5 MPa, preferably in the range 2.0 MPa to 3.5 MPa. The hydrogen flow rate is adjusted in order to have it available in sufficient quantity for theoretical hydrogenation all of the diolefins, acetylenes and aromatic alkenyl compounds and to maintain an excess of hydrogen at the reactor outlet. In order to limit the temperature gradient in the reactor, it may be advantageous to recycle a fraction of the effluent to the inlet and/or to the mid part of the reactor.

The following examples illustrate the invention without limiting the scope. The attrition test carried out on the materials prepared in Examples 1 to 5 below were carried out under the conditions described above in the present description using the Spence method using a NFX-11-504/ISO-3310-2 normalized sieve with an opening diameter of 1 mm.

### EXAMPLE 1 (Invention)

### Preparation of A Core-Layer Material

This example describes the preparation of a material containing a core of alpha alumina and a layer of gamma alumina. The layer was formed on a pre-layer from a powder formed by spherical particles with a calibrated size distribution, an aqueous suspension containing an organic binder and an inorganic binder with the addition of hot air. The pre-layer was formed from the same suspension as the layer.

The core was prepared from spherical particles of gamma alumina with reference Spheralite 537 (Axens). These beads were calcined at 1200° C. to transform them into alpha alumina and had a specific surface area of 9 m²/g, a total pore volume of 0.28 mL/g and a mean pore size of 90 nm according to nitrogen physisorption analysis (ASAP2420, Micromeretics). TEM analysis (Supra, Zeiss) showed perfectly spherical particles with a smallest diameter of 1.45 mm; the largest diameter was 1.75 mm and the mean diameter was 1.6 mm.

The pre-layer was formed directly on the core from an aqueous suspension of boehmite peptized in nitric acid containing polyvinyl alcohol. The suspension was prepared by dispersing a commercially available boehmite with reference Pural SB3 (Sasol) in a nitric acid solution in order to obtain a HNO₃/AlOOH ratio of 3.35% and a AlOOH/(AlOOH+H₂O) weight ratio of 3%. The mixture was agitated at ambient temperature for 2 h. The suspension was centrifuged at 3800 g for 20 min to extract all of the non-peptized boehmite. Polyvinyl alcohol (Carlo Erba) was dissolved in the suspension in order to obtain a PVA/AlOOH ratio of 3% by weight. The suspension was deposited on the alpha alumina particles in a laboratory pan granulator (Grelbex P30) equipped with a cylindro-conical plate executing a cascade type movement of the bed of beads constituting the core. To this end, 100 g of alpha alumina particles were placed in the plate inclined at 30° and rotated at 40 rpm and 28 mL of suspension was sprayed at a mean flow rate of 1 mL/min over said particles resulting in a AlOOH/α-Al₂O₃ weight ratio of 0.86%.

The layer was then formed directly on the pre-layer around the core starting from a gamma alumina powder and an aqueous suspension of boehmite peptized in nitric acid containing poly vinyl alcohol, in a manner identical to that prepared for the pre-layer. The gamma alumina powder was prepared by drying by atomizing an aqueous suspension containing a boehmite (Pural SB3, Sasol), nitric acid and aluminium nitrate then by calcining. The gamma alumina particles forming the powder were spherical according to the TEM analysis. The characteristic sizes measured by laser diffraction granulometry (Mastersizer 2000, Malvern) were: Dv50=2 μm, Dv10=1 μm and Dv90=3 μm. The powder has a specific surface area of 223 m²/g, a pore volume of 0.35 mL/g and a median pore size of 7 nm, according to the nitrogen physisorption analysis. Deposition of the layer on the alpha alumina particles provided with a pre-layer was carried out in the same pan granulator executing a cascade movement of the bed of particles containing the pre-layer. 6 g of gamma alumina powder were introduced progressively at a mean flow rate of 0.13 mL/min onto the particles containing the pre-layer resulting in a γ Al₂O₃/α Al₂O₃ weight ratio of 6%. 61 mL of suspension was simultaneously sprayed at a mean flow rate of 1 mL/g onto the particles containing the pre-layer, resulting in a mass/volume AlOOH/γ Al₂O₃ ratio of 0.24 g/mL. The PVA/AlOOH weight ratio was identical to that for the step for the formation of the pre-layer and was equal to 3%. A continuous stream of air at 70° C. was simultaneously applied to the bed of spherical particles provided with the pre-layer.

The material obtained after depositing the layer was dried in a ventilated oven at 100° C., for 2 h in ambient air then calcined in a muffle furnace at 600° C. for 2 h in ambient air.

TEM analysis (Supra, Zeiss) of a polished section of material showed a continuous layer with a homogeneous thickness of 18 μm to 22 μm, with a mean value of 20 μm. The TEM analysis of the polished material section also showed that the particles of gamma alumina forming the powder were very close or touching and that the interparticulate voids were filled with inorganic binder with no residual porosity, crazing or crack type defect. The inorganic binder was constituted by gamma alumina resulting from the conversion of boehmite following the calcining step. TEM analysis of the pre-layer, carried out after carrying out calcining step d) without carrying out step b) for formation of the layer coating the core, indicated a thickness on the outer surface of 1 μm and a depth of 1.5 μm. The degree of attrition of the material was very low, equal to 2.7%, and was minimal for this quantity of boehmite introduced. The attrition resistance was thus very high and satisfactory.

### EXAMPLE 2 (Invention)

### Preparation of A Core-Layer Material

This example describes the preparation of a material containing a core of alpha alumina and a layer of gamma alumina. The layer was formed from a powder of spherical particles with a calibrated size distribution, an aqueous suspension containing an organic binder and an inorganic binder with the addition of hot air. The pre-layer was formed from the same suspension as the layer with no addition of organic binder.

The conditions for preparation, the operating protocol and the reagents used were identical to those described in Example 1, but with no addition of polyvinyl alcohol to the suspension intended for the formation of the pre-layer.

TEM analysis (Supra, Zeiss) of a polished section of material showed a continuous layer with a homogeneous thickness of 17 μm to 23 μm. The TEM analysis of the polished material section also showed that the particles of gamma alumina forming the powder were very close or touching and that the interparticulate voids were filled with inorganic binder constituted by y alumina following the calcining step, with no residual porosity or crazing or cracking defects. TEM analysis of the pre-layer, carried out after carrying out calcining step d) without carrying out step b) for the formation of the layer coating the core on a polished section, indicated a thickness on the outer surface of 1 μm and a depth of 1 μm. The degree of attrition of the material was very low, equal to 3.5%. The attrition resistance was thus very high and satisfactory.

### EXAMPLE 3 (Comparative)

### Preparation of A Core-Layer Bimaterial Free of Pre-Layer

This example describes the preparation of a material containing a core of alpha alumina and a layer of gamma alumina. Said material was free of pre-layer. The layer was formed from a powder of spherical particles with a calibrated size distribution, an aqueous suspension containing an organic binder and an inorganic binder with the addition of hot air.

The preparation conditions, the operating protocol and the reagents used were identical to those in Example 1 but without the step for pre-layer formation.

TEM analysis (Supra Zeiss) of a polished section of material showed a layer which in some locations was fragmented, non-continuous and non-homogeneous as regards thickness and at other locations it showed the gamma alumina particles forming the powder close to each other and wherein the interparticulate voids were filled with inorganic binder with no residual porosity, but with many crazing and cracking type defects at the layer-core interface resulting in the fragmentation. The degree of attrition of the material was very high and equal to 90%. The attrition resistance was thus extremely low and unsatisfactory.

### EXAMPLE 4 (Comparative)

### Preparation of A Core-Layer Material With the Layer Constituted By Non-Spherical Particles

This example describes the preparation of a material containing a core of alpha alumina and a layer of gamma alumina. The layer was formed on a pre-layer from a powder formed by non-spherical particles with a calibrated size distribution, from an aqueous suspension containing an organic binder and an inorganic binder with the addition of hot air. The pre-layer was formed from the same suspension as the layer.

The preparation conditions, the operating protocol and the reagents used were identical to those in Example 1 but with the use of a powder formed from particles of non-spherical gamma alumina and a sprayed volume of boehmite suspension slightly higher in step b) for the formation of a layer coating the core to fill the interparticulate voids formed by the powder particles.

The gamma alumina powder was prepared by air jet spraying of the powder with reference Puralox (Sasol). The particles had a highly variable shape, derived from fragmentation, according to TEM analysis. The characteristic sizes measured by laser diffraction granulometry (Mastersizer 2000, Malvern) were as follows: Dv50=2 μm, Dv10=1 μm and Dv90=4 μm. The powder had a specific surface area of 210 m²/g, a pore volume of 0.35 mL/g and a median pore size of 8 nm, according to the nitrogen physisorption analysis (ASAP 2420, Micromeretics). The volume of the boehmite suspension of 70 mL was sprayed progressively at a mean flow rate of 1.15 mL/min and resulted in a AlOOH/γ Al₂O₃ mass/volume ratio of 0.27 g/mL.

TEM analysis (Supra Zeiss) of a polished section of material showed a layer which in certain locations was slightly fragmented, discontinuous and of poor homogeneity as regards thickness and at other locations it showed the gamma alumina particles forming the powder being relatively close to each other and wherein the interparticulate voids were filled with inorganic binder, but many crazing and cracking type defects were present in the binder, leading to the observed fragmentation. The degree of attrition of the material was 20%. The attrition resistance was thus low and unsatisfactory.

### EXAMPLE 5 (Comparative)

### Preparation of A Core-Layer Material

This example describes the preparation of a material containing a core of alpha alumina and a layer of gamma alumina. The layer was formed on a pre-layer from a powder formed by spherical particles with a calibrated size distribution from an aqueous suspension containing an organic binder and an inorganic binder with the addition of hot air. The pre-layer was formed from the same suspension as the layer.

The preparation conditions, the operating protocol and the reagents used were identical to those in Example 1 but with a very high volume of boehmite suspension to form a layer coating the core which did not satisfy the mass/volume AlOOH/γ Al₂O₃ ratio recommended in the invention.

The volume of the boehmite suspension of 331 mL was sprayed progressively at a mean flow rate of 5.43 mL/min and resulted in a AlOOH/γ Al₂O₃ mass/volume ratio of 1.3 g/mL.

TEM analysis of the material showed a completely fragmented, discontinuous and non-homogeneous layer as regards its thickness as well as particles of gamma alumina forming the powder which were relatively distanced from each other and an inorganic binder comprising many crazing and cracking type defects resulting in total fragmentation. The degree of attrition of the material was 95%. The attrition resistance was thus extremely low and unsatisfactory.

### EXAMPLE 6 (Invention)

### Preparation of A Catalyst A

25 g of material prepared in accordance with Example 1 was dry impregnated in a pan granulator at 25° C. with an aqueous solution of palladium nitrate Pd(NO₃)₂. Said solution was prepared by diluting 0.54 g of an aqueous 10% by weight palladium nitrate solution and 10% by weight of nitric acid (Aldrich) in demineralized water to a total volume which corresponded to the pore volume of said material. No attrition of said material was observed during the impregnation step.

The catalyst A obtained was dried in air at 120° C. then calcined for 2 hours at 450° C. in air. The catalyst A contained 0.1% by weight of palladium deposited in the porosity of the layer of material prepared in accordance with Example 1.

### EXAMPLE 7 (Comparative)

### Preparation of A Catalyst B

25 g of material prepared in accordance with Example 3 was dry impregnated in a pan granulator at 25° C. with an aqueous solution of palladium nitrate Pd(NO₃)₂. Said solution was prepared by diluting 0.54 g of an aqueous 10% by weight palladium nitrate solution and 10% by weight of nitric acid (Aldrich) in demineralized water to a total volume which corresponded to the pore volume of said material. The material was subjected to attrition during the impregnation step.

The quantity of fines generated was weighed and estimated to be approximately 60% of the mass of the layer initially present in said material.

The catalyst B obtained was dried in air at 120° C. then calcined for 2 hours at 450° C. in air. The catalyst B contained 0.1% by weight of palladium deposited in the porosity of the layer of material prepared in accordance with Example 3.

### EXAMPLE 8 (Comparative)

### Preparation of A Catalyst C

25 g of material prepared in accordance with Example 4 was dry impregnated in a pan granulator at 25° C. with an aqueous solution of palladium nitrate Pd(NO₃)₂. Said solution was prepared by diluting 0.54 g of an aqueous 10% by weight palladium nitrate solution and 10% by weight of nitric acid (Aldrich) in demineralized water to a total volume which corresponded to the pore volume of said material. No attrition of said material was observed during the impregnation step.

The catalyst C obtained was dried in air at 120° C. then calcined for 2 hours at 450° C. in air. The catalyst C contained 0.1% by weight of palladium deposited in the pores of the layer of material prepared in accordance with Example 4.

### EXAMPLE 9

### Catalytic Test For Selective Hydrogenation of A Styrene-Isoprene Mixture

Before the catalytic test, catalysts A, B and C were treated in a stream of 1000 litres of hydrogen per hour per litre of catalyst with a temperature ramp-up of 300° C./h and a constant temperature stage at 150° C. of 2 hours.

The catalysts then successively underwent a hydrogenation test in a perfectly stirred discontinuous reactor of the “Grignard” type. To this end, 2 mL of reduced catalyst beads were fixed, with air being excluded, in an annular basket located around the agitation actuator. The baskets used in the reactors were of the Robinson Mahonnay type.

Hydrogenation was carried out in the liquid phase.

The composition of the feed was as follows: 8% by weight of styrene, 8% by weight of isoprene, the solvent being n-heptane. This feed was a model feed for pyrolysis gasoline.

The test was carried out at a constant pressure of 3.5 MPa of hydrogen and at a temperature of 45° C. The products of the reaction were analyzed by gas chromatography. The catalytic activities were expressed as moles of H₂ consumed per minute and per gram of palladium and are reported in Table 1.

TABLE 1
activities measured for the hydrogenation
of a styrene-isoprene mixture

| Catalyst | Activity* |
|---|---|
| Catalyst A (invention) | 8.06 |
| Catalyst B (comparative) | 1.33 |
| Catalyst C (comparative) | 3.76 |

*in (moles H₂)/[min × (grams of palladium)]

Catalyst A prepared from the core-layer material with a low degree of attrition and with a continuous layer and homogeneous thickness prepared in accordance with the preparation process of the invention was substantially more active than catalyst B prepared from a core-layer material with an extremely low attrition resistance and with a non-continuous and non-homogeneous layer as regards thickness. This same catalyst A was also substantially more active than catalyst C prepared from a core-layer material with a low attrition resistance and having a non-continuous and non-homogeneous layer as regards thickness.

1. A process for preparing a spherical material comprising at least one porous core coated with at least one continuous and homogeneous porous layer, the degree of attrition of said material, measured using the Spence method, being less than 20%, said preparation process comprising at least the following steps:
a) bringing at least one bed of spherical particles constituting the core of said material into contact with at least one suspension containing at least one inorganic binder in order to form a solid having a pre-layer surrounding said core;
b) bringing the solid derived from said step a) into contact in a stream of hot air with at least one powder constituted by spherical particles of at least one inorganic oxide and at least one suspension containing at least one inorganic binder and at least one organic binder in order to form a solid the core of which is coated with at least one continuous and homogeneous porous layer, the ratio of the (mass of anhydrous inorganic binder/volume of powder particles) being in the range 0.05 to 1 g.mL⁻¹;
c) drying the solid derived from said step b);
d) calcining the solid derived from said step c).

2. A preparation process according to claim 1, wherein the core is constituted by an alpha alumina and the layer by a gamma alumina.

3. A preparation process according to claim 1, wherein the thickness of the layer has a mean value in the range 0.005 to 1 mm.

4. A preparation process according to claim 1 wherein said material has a degree of attrition of less than 5%.

5. A preparation process according to claim 1, wherein the suspension used to carry out said step a) contains at least one organic binder.

6. A preparation process according to claim 1, wherein said step a) is carried out in the absence of any flow of air.

7. A preparation process according to claim 1, wherein said step a) is carried out by spraying said suspension onto said bed of spherical particles contained in an inclined rotating plate or in a rotating drum and executing a cascade type movement.

8. A preparation process according to claim 1 wherein the ratio of the (mass of anhydrous inorganic binder/volume of particles of powder) ratio in said step b) is in the range 0.15 to 0.35 g.mL⁻¹.

9. A preparation process according to claim 1, wherein the temperature of the hot air stream during said step b) is in the range 60° C. to 150° C.

10. A process for preparing a catalyst, comprising at least the following steps:
e) impregnating material prepared in accordance with claim 1 with at least one solution of at least one precursor of at least one metal from group VIII;
f) drying the solid derived from said step e);
g) calcining the solid derived from said step f); and
h) reducing the catalyst derived from said step g).

11. A process for the preparation of a catalyst according to claim 10, wherein said metal from group VIII is palladium.

12. A process for the preparation of a catalyst according to claim 10 wherein, following said step f) a step e′) for impregnation of said solid derived from said step f) is carried out with at least one solution of at least one precursor of at least one metal from group IB.

13. A process for selective hydrogenation comprising bringing a hydrocarbon feed containing at least one polyunsaturated compound into contact with at least one catalyst prepared using the preparation process according to claim 10.

14. A selective hydrogenation process according to claim 13, wherein said hydrocarbon feed is selected from the group constituted by cuts derived from catalytic cracking, the C3 cut derived from steam cracking, the C4 cut derived from steam cracking, the C5 cut derived from steam cracking and gasolines derived from steam cracking.
